(19) United States
(12) Patent Application Publication
(10) Pub. No.: US 2006/0223816 A1
(43) Pub. Date: Oct. 5, 2006

(54) Imatinib mesylate alpha form and production process therefor

(75) Inventors: Itai Adin, Beer Sheva (IL); Carmen Iustain, Beer Sheva (IL); Guy Davidi, Even Yehuda (IL); Alex Weisman, Kiriyat Ekron (IL); Moshe Bentolila, Moshav Tekoma (IL); Elazar Meyer, Matta (IL); Joseph Kaspi, Givatayim (IL)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: CHEMAGIS LTD., Bnei Brak (IL)

(21) Appl. No.: 11/429,731

(22) Filed: May 8, 2006

(51) Int. Cl. A61K 31/506; C07D 403/14

(52) U.S. Cl. 514/252.18; 544/295

(57) ABSTRACT

Provided is a process for preparing crystalline imatinib mesylate in substantially pure α-form, which preferably includes crystallizing imatinib mesylate from an organic solvent containing imatinib and methanesulfonic acid, and seed crystals of imatinib mesylate α-form, wherein the seed crystals are added before imatinib mesylate begins to precipitate from the mixture. Also provided are stable, free-flowing imatinib mesylate crystals in substantially pure α-form, and a pharmaceutical composition containing the stable, free-flowing imatinib mesylate crystals.

### BACKGROUND OF THE INVENTION

Imatinib (N-{5-[4-(4-methyl-piperazinomethyl)-benzoylamido]-2-methylphenyl}-4-(3-pyridyl)-2-pyrimidine-amine) is represented by the following structural formula (I):

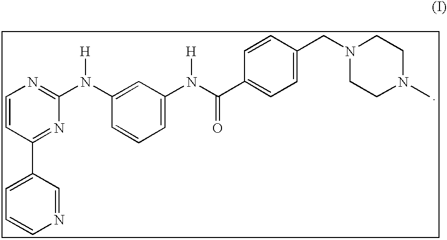

Imatinib is known as an inhibitor of tyrosine kinases and is indicated for the treatment of chronic myeloid leukemia (CML), Philadelphia chromosome positive leukemia, for patients in chronic phase and in blast crisis, accelerated phase and also for malignant gastrointestinal stromal tumors. It selectively inhibits activation of target proteins involved in cellular proliferation. Imatinib also has potential for the treatment of other cancers that express these kinases, including acute lymphocytic leukemia and certain solid tumors. Imatinib is sold by Novartis as Gleevec™ capsules containing imatinib mesylate equivalent to 100 mg of imatinib free base.

U.S. Pat. No. 6,894,051 (“the '051 patent”) describes two crystalline forms of imatinib mesylate, the α-form and the β-form. The '051 patent teaches that the α-form is hygroscopic and that it is characterized by needle-shaped crystals, which make them “not particularly well-suited to pharmaceutical formulation as solid dosage forms, because their physical properties, for example their flow characteristics, are unfavorable.” Example 1 of the '051 patent describes a process for preparing the α-form, which includes suspending imatinib base in ethanol, adding methanesulfonic acid, heating to reflux and filtering to obtain a filtrate, evaporating down to 50% of its original volume, filtering off the residue, evaporating the mother liquor to dryness, suspending the resulting residue and the filtrate in ethanol, dissolving under reflux with the addition of water, cooling the product overnight, and obtaining the product by filtration. This process is cumbersome since it involves evaporation of both the filtrate and the mother liquor. The '051 patent also teaches that imatinib mesylate transformation of the α-form into the β-form can occur spontaneously in solution. The '051 patent also describes a process for obtaining the β-crystalline form from the α-form by digesting the α-form in methanol at 25° C. for two days.

WO 2004/106326 (“the '326 application”) describes a crystalline form of imatinib mesylate, designated as form H1, and processes for obtaining this form. The '326 application teaches producing the imatinib mesylate designated as form H1 from chlorinated solvents such as chloroform and dichloromethane. Using chlorinates solvents is not particularly desirable for industrial implementation due to the hazards associated with such solvents.

WO 2005/095379 (“the '379 application”) describes a method of preparing the α-form using a reduced molar ratio of methanesulfonic acid, which is 0.95-0.99 moles of methanesulfonic acid per mole of imatinib, in the reaction mixture. The method described in the '379 application generally includes adding methanesulfonic acid to a solution of imatinib in an alcohol or a mixture of alcohol and ester, cooling, and seeding at temperatures close to the temperature of crystallization (i.e., after completing the addition of methanesulfonic acid and after cooling), and further cooling and filtering. However, this process is not necessarily reproducible or viable on an industrial scale.

WO 2006/024863 (“the '863 application”) also describes a method of preparing crystalline imatinib mesylate α-form; however, the '863 application teaches micronizing the product order to change the undesirable crystalline needle form and obtain desirable physical properties of the solid.

In view of the limitations associated with the α-form and methods of producing the α-form, there is a need for a refined α-form of imatinib mesylate, which exhibits excellent physical properties without the need for micronization, and a simple, reproducible and straightforward method of producing such a product, which can be carried out using safe solvents. The present invention provides such a product and method.

### SUMMARY OF THE INVENTION

The Applicants have surprisingly discovered that a stable, free-flowing imatinib mesylate α-form, which is substantially free of the β-form, can be reproducibly obtained by seeding with imatinib mesylate α-form seed crystals before imatinib mesylate begins to precipitate from the solution, preferably before or during the addition of methanesulfonic acid. The process of the present invention produces a refined form of crystalline imatinib mesylate α-form, which is free-flowing and suitable for pharmaceutical compositions, and yet does not need to be micronized and can be produced using a simple, straight-forward procedure using industrially safe solvents.

The process of the present invention preferably includes crystallizing imatinib mesylate from a solution comprising an organic solvent, with imatinib and methanesulfonic acid dissolved therein, and seed crystals of imatinib mesylate in substantially pure α-form, wherein the seed crystals are added before imatinib mesylate begins to precipitate from the solution. In one embodiment of the present invention, seeding is carried out before the addition of methanesulfonic acid or at the beginning of the acid addition phase, but sufficiently in advance of the time that solid imatinib mesylate begins precipitating from solution. Without wishing to be bound by any particular theory, it is believed that seeding prior to precipitation (e.g., prior to cooling) may prevent the formation of imatinib mesylate β-form, e.g., so that the crystallization that follows the addition of the acid is controlled by the presence of α-form seeds, such that the crystalline β-form is not created at all.

In one embodiment, the present invention provides a reproducible process for preparing a stable, free-flowing form of crystalline imatinib mesylate α-form, which process includes: mixing imatinib base with an organic solvent and heating, e.g., to dissolve some or substantially all of the imatinib base in the organic solvent; adding methanesulfonic acid, e.g., by preparing a solution of methanesulfonic acid in the organic solvent; seeding with imatinib mesylate α-form; gradually (e.g., slowly) adding the methanesulfonic acid (e.g., as a solution of methanesulfonic acid in the organic solvent) to the mixture of imatinib base and the organic solvent; allowing crystals of imatinib mesylate to precipitate (e.g., by allowing the solution to cool); and isolating the precipitated crystals of imatinib mesylate α-form.

In another embodiment, the present invention provides a reproducible process for preparing a stable, free-flowing form of crystalline imatinib mesylate α-form, which process includes: mixing imatinib base with an organic solvent and heating, e.g., wherein at least a portion of the imatinib base dissolves or exists as a suspension in the organic solvent; preparing a solution of methanesulfonic acid in the organic solvent; gradually (e.g., slowly) adding about one third of the volume of the solution of methanesulfonic acid in the organic solvent to the mixture of imatinib base and the organic solvent; seeding with imatinib mesylate α-form; gradually (e.g., slowly) adding the remaining about two thirds of the volume of the solution of methanesulfonic acid in the organic solvent to the mixture of imatinib base and the organic solvent; allowing crystals of imatinib mesylate to precipitate (e.g., by allowing the solution to cool); and isolating the precipitated crystals of imatinib mesylate α-form.

Suitable organic solvents, which can be used to obtain imatinib mesylate α-form in accordance with the process of the present invention, include, e.g., methyl ethyl ketone (MEK), methyl isobutyl ketone (MIBK), cyclohexanone, 4-methylcyclohexanone, acetonitrile, and mixtures thereof.

The present invention additionally provides imatinib mesylate α-form in a stable and free-flowing form, which is suitable for use in pharmaceutical compositions without the need for micronization. The stable, free-flowing imatinib mesylate α-form of the present invention is substantially free of imatinib mesylate β-form. The present invention also provides a composition that includes a pharmaceutically acceptable carrier and a therapeutically effective amount of the stable, free-flowing imatinib mesylate α-form of the present invention.

### BRIEF DESCRIPTION OF THE DRAWINGS

FIG. 1 depicts the DSC curve of the product obtained as per example 1 of the '051 patent for preparing imatinib mesylate α-form.

FIG. 2 depicts the infra-red spectrum of the product obtained as per example 1 of the '863 application.

FIG. 3 depicts the DSC curve of the product obtained as per example 1 of the '863 application.

### DETAILED DESCRIPTION OF THE INVENTION

The Applicants have surprisingly discovered that it is possible to reproducibly prepare a stable, free-flowing imatinib mesylate α-form, which is substantially free of the β-form, by seeding with imatinib mesylate α-form seed crystals prior to precipitation of the product, preferably before or some time after the addition of at least a portion of methanesulfonic acid. In one respect, the present invention provides a process for preparing crystalline imatinib mesylate in substantially pure α-form, which process includes crystallizing imatinib mesylate from a solution of imatinib base and methanesulfonic acid in an organic solvent containing seed crystals of imatinib mesylate α-form, wherein the seed crystals are added before imatinib mesylate begins to precipitate from the solution.

Preferably, seeding is carried out before the addition of methanesulfonic acid or at the beginning of the acid addition process, but with enough time before the precipitation of solid imatinib mesylate so the crystallization that follows the addition of the acid is controlled by the presence of the α-form seeds such that the crystalline β-form is not formed to any appreciable extent, e.g., not at all. More preferably, the seeding is carried out prior to the cooling, e.g., before or some time after starting the addition of methanesulfonic acid.

In accordance with the present invention, any suitable quantity of seed crystals can be used. An exemplary weight ratio between the seed crystals of imatinib mesylate α-form to imatinib base in the reaction mixture is about 5% (e.g., 50 mg of imatinib mesylate α-form per 1 g of imatinib base).

In one embodiment, the present invention provides a reproducible process for preparing a stable, free-flowing form of crystalline imatinib mesylate in substantially pure α-form, which includes: mixing imatinib base with an organic solvent and heating, e.g., to dissolve at least a portion of or substantially all of the imatinib base in the organic solvent; adding methanesulfonic acid (e.g., as a solution of methanesulfonic acid in the organic solvent); seeding with imatinib mesylate α-form seed crystals; gradually (e.g., slowly) adding methanesulfonic acid (e.g., as a solution of methanesulfonic acid in the organic solvent) to the mixture of imatinib base and the organic solvent; precipitating crystals of imatinib mesylate (e.g., by allowing the mixture to cool); and isolating the precipitated crystals of imatinib mesylate α-form.

In another embodiment, the present invention provides a reproducible process for preparing a stable, free-flowing form of crystalline imatinib mesylate in substantially pure α-form, which process includes: mixing imatinib base with an organic solvent and heating, e.g., wherein at least a portion of the imatinib base dissolves or exists as a suspension in the organic solvent; providing a solution of methanesulfonic acid in the organic solvent and gradually (e.g., slowly) adding about one third of the volume of the solution of methanesulfonic acid in the organic solvent to the mixture of imatinib base and the organic solvent; seeding with imatinib mesylate α-form seed crystals; gradually (e.g., slowly) adding the remaining about two thirds of the volume of the methanesulfonic acid solution to the mixture of imatinib base and the organic solvent; precipitating crystals of imatinib mesylate (e.g., by allowing the mixture to cool); and isolating the precipitated crystals of imatinib mesylate α-form.

Suitable organic solvents, which can be used to obtain imatinib mesylate α-form in accordance with the present invention, include methyl ethyl ketone (MEK), methyl isobutyl ketone (MIBK), cyclohexanone, 4-methyl-cyclohexanone, acetonitrile, and mixtures thereof. The organic solvent(s) preferably include(s) one or more class 3 solvents e.g., MEK or MIBK, which enable precipitating imatinib mesylate α-form upon completion of the addition of methanesulfonic, or non-chlorinated class 2 solvent e.g., acetonitrile. According to industrial guidelines on residual solvents, last issued on 1997 (Appendixes 5-7: toxicological data for class 1-3 solvents respectively), published by the International Conference on Harmonization (ICH), the use of industrial solvents is restricted according to their safety features. The industrial solvents are divided into three main classes:

Class 1: Solvents that should not be employed in the manufacture of drug substances or drug products because of their unacceptable toxicity or their deleterious environmental effect. Solvents that belong to this class are: benzene, carbon tetrachloride, 1,2-dichloroethane and others.

Class 2: Solvents that should be limited in pharmaceutical products because of their inherent toxicity. Important industrial solvents that belong to this class are chlorinated solvents such as chloroform, hydrocarbons such as hexane and aromatic solvents such as toluene.

Class 3: Solvents that are regarded as less toxic and of lower risk to human health. Important industrial solvents that belong to this class are certain ketones, esters, alcohols and others.

The solvent 4-methylcyclohexanone, which can be used in the process of the present invention, belongs to a group of flavoring agents that are permitted to be used in foods, hence there is no safety concern while using it as such at current level of intake, as determined in the toxicological monograph FAS 50-JECFA 59/331.

The process of the present invention does not require reducing the molar quantity of methanesulfonic acid, e.g., does not require limiting the methanesulfonic acid to 0.95 moles of acid per mole of imatinib base, as suggested in the '379 application, since the process of the present invention allows the imatinib mesylate to be precipitated during the addition of the methanesulfonic salt, hence, the final molar ratio of methanesulfonic acid to imatinib base is not critical. It should be apparent to those of ordinary skill in the art that using a lower molar quantity of methanesulfonic acid may lower the yield of obtaining the crystalline imatinib mesylate accordingly. Preferably, the molar ratio of imatinib base:methanesulfonic acid used in the process of the present invention is about 1:1.

The process of the present invention further can prevent the transition of the resulting α-form crystals into another form, e.g., the imatinib mesylate β-form. According to one aspect of the present invention, the formation of the β-form crystals is not observed even after incubating the crystallization mixture, containing the α-form crystals, for prolonged periods in the reaction vessel overnight, as determined by using the DSC technique. For instance, it is possible to carry out the process of the present invention without observing the formation of the β-form crystals even after incubating the crystallization mixture, containing the α-form crystals (and even an excess of methanesulfonic acid), in the reaction vessel overnight (e.g., after about 15 hours), as determined by using the DSC technique.

In addition, the process of the present invention can be performed without complete dissolution of imatinib base, e.g., wherein a suspension of imatinib base in the appropriate solvent is prepared (e.g., wherein at least a portion of the imatinib base is suspended in the organic solvent), which is seeded with pure imatinib mesylate α-form, and then methanesulfonic acid is slowly added as described herein.

Preferably, the process of the present invention is performed at a temperature, which is sufficiently low to avoid substantial thermal degradation of the imatinib base, which is believed to be heat sensitive. Preferably, the process of the present invention is carried out at a temperature which is lower than 80° C., and more preferably at a temperature which is lower than 70° C., when ketone solvents are used, e.g., methyl ethyl ketone (MEK), methyl isobutyl ketone (MIBK), and 4-methyl-cyclohexanone. Alternatively, the process of the present invention is preferably carried out at a temperature which is equal to or lower than 40° C., and more preferably at a temperature equal to or lower than 15° C., when acetonitrile is used.

The identification of the crystalline form of imatinib mesylate can be performed by any suitable method, including traditional solid-state techniques e.g., infra-red spectroscopy (IR), differential scanning calorimetry (DSC) and X-ray powder diffraction (XRPD).

The present invention additionally provides an imatinib mesylate α-form as a stable and free-flowing solid, which is suitable for pharmaceutical compositions without the need to micronize the crystals, making this form particularly suitable for pharmaceutical applications. The imatinib mesylate α-form is substantially free of imatinib mesylate β-form and can be obtained in accordance with the present invention in at least about 86.5% yield, e.g., in at least about 92% yield. Further, the imatinib mesylate α-form of the present invention has a purity of at least about 98.8%, e.g., a purity of about 99.5% or higher.

The present invention further provides a composition that includes a pharmaceutically acceptable carrier and a therapeutically effective amount of the stable, free-flowing, substantially pure α-form imatinib mesylate of the present invention.

### EXAMPLES

The following examples further illustrate the invention but, of course, should not be construed as in any way limiting its scope.

The examples describe processes for preparing pure imatinib mesylate α-form, wherein the term “pure” refers to a product that is substantially free of other crystalline forms (e.g., the imatinib mesylate β-form or a di-mesylate). The purity of imatinib mesylate α-form obtained thereby was observed using one or more known solid-state techniques.

General description of the equipment:

X-ray diffraction data were acquired using a PHILIPS X-ray diffractometer model PW1050-70. System description: K_{α1}=1.54178Å, voltage 40 kV, current 28 mA, diversion slit−1°, receiving slit=0.2mm, scattering slit=1° with a Graphite monochromator. Measurements of 2θ values typically are accurate to within ±0.2 degrees. Experiment parameters: pattern measured between 2θ=3° and 2θ=30° with 0.05° increments; count time was 0.5 second per increment.

Infra-red spectra were run on Nicolet Fourrier-transform infra-red spectrometer model Avatar 360, with Omnic software version 5.2. All samples were run as KBr disks. The current infra-red measurements are accurate to within 4 cm⁻¹.

Differential scanning calorimetry (DSC) measurements were run on TA instruments model Q1000, with Universal software version 3.88. Samples were analyzed inside crimped 40 μl Aluminum pans. Heating rate for all samples was 10° C./min.

### Example 1

A three-necked reaction vessel equipped with a thermometer, a reflux condenser and a mixer was charged with 0.505 gram of imatinib base (1.02 mmoles) under nitrogen atmosphere and mixed with 48 ml of methyl ethyl ketone. The mixture was heated to 75-77° C. until a clear solution was obtained, which was seeded with 25 mg of imatinib mesylate α-form. 69 μL (1.02 mmoles) of methanesulfonic acid were mixed with 5 ml of methyl ethyl ketone to form a solution, followed by slow addition of the thus formed methanesulfonic acid solution to the seeded solution of imatinib base during 2 hours. At the end of the addition the thus formed suspension was cooled to room temperature and the resulting crystals were filtered and dried under reduced pressure to obtain 0.51 g of imatinib mesylate α-form in 86.5% yield. The purity was determined by HPLC (98.8%).

### Example 2

A three-necked reaction vessel equipped with a thermometer, a reflux condenser and a mixer was charged with 0.505 gram (1.02 mmoles) of imatinib base under nitrogen atmosphere and mixed with 48 ml of methyl ethyl ketone. The mixture was heated to 75-77° C. until a clear solution was obtained. 69 μL (1.02 mmoles) of methanesulfonic acid were mixed with 5 ml of methyl ethyl ketone, followed by slow addition of the acid solution during 2 hours. After addition of 30% of the acid the solution, which was still clear, was seeded with 25 mg of imatinib mesylate α-form. At the end of the addition the thus formed suspension was cooled to room temperature and the resulting crystals were filtered and dried under reduced pressure to obtain 0.52 g of imatinib mesylate α-form in 88% yield.

### Example 3

A three-necked reaction vessel equipped with a thermometer, a reflux condenser and a mixer was charged with 1.01 gram of imatinib base (2.05 mmoles) under nitrogen atmosphere and mixed with 20 ml of methyl isobutyl ketone. The mixture was heated to 65° C. and seeded with 50 mg of imatinib mesylate α-form. 375 μL of methanesulfonic acid were mixed with 30 ml of methyl isobutyl ketone to form a solution, and 11.1 ml out of this solution (2.05 moles) were slowly added to the seeded solution of imatinib base during 4 hours. At the end of the addition the thus formed suspension was cooled to room temperature and the resulting wet crystals were filtered and dried under reduced pressure to obtain 1.085 g of imatinib mesylate α-form in 92% yield. The purity was determined by HPLC (99.4%).

### Example 4

A three-necked reaction vessel equipped with a thermometer, a reflux condenser and a mixer was charged with 1.004 grams of imatinib mesylate (2.04 mmoles) under nitrogen atmosphere and mixed with 20 ml of methyl ethyl ketone. The mixture was heated to 65° C. and seeded with 50 mg of imatinib mesylate α-form. 375 μl of methanesulfonic acid were dissolved in 30 ml of methyl ethyl ketone, and 11 ml out of this solution (2.04 mmoles) were slowly added to the imatinib mixture during 4 hours. The thus formed suspension was cooled to room temperature and the resulting wet crystals were filtered and dried under reduced pressure. The purity was determined by HPLC (99.5%).

### Example 5

A three-necked reaction vessel equipped with a thermometer, a reflux condenser and a mixer was charged with 1.004 gram of imatinib base (2.04 mmoles) under nitrogen atmosphere and mixed with 40 ml of acetonitrile. The mixture was cooled to 15° C. and seeded with 50 mg of imatinib mesylate α-form. 0.375 ml of methanesulfonic acid was mixed with 30 ml of acetonitrile, and 11 ml (2.04 mmoles) out of this solution were slowly added to the imatinib mixture during 5 hours. The thus formed suspension was filtered and dried under reduced pressure to obtain 1.065 g of imatinib mesylate α-form in 90% yield. The purity was determined by HPLC (99.4%).

### Example 6

A three-necked reaction vessel equipped with a thermometer, a reflux condenser and a mixer was charged with 1.004 gram of imatinib base (2.04 mmoles) under nitrogen atmosphere and mixed with 30 ml of 4-methylcyclohexanone. The mixture was heated to 65° C. and seeded with 50 mg of imatinib mesylate α-form. 375 μL of methanesulfonic acid were mixed with 30 ml of 4-methylcyclohexanone to form a solution, and 11 ml (2.04 mmoles) of the thus formed methanesulfonic acid solution was slowly added to the seeded solution of imatinib base during 4 hours. At the end of the addition the obtained suspension was cooled to room temperature and the resulting wet crystals of imatinib mesylate α-form were filtered and dried. The purity by HPLC was 99.6%.

### Comparative Example 1

This example illustrates an attempt to repeat example 1 of the '051 patent.

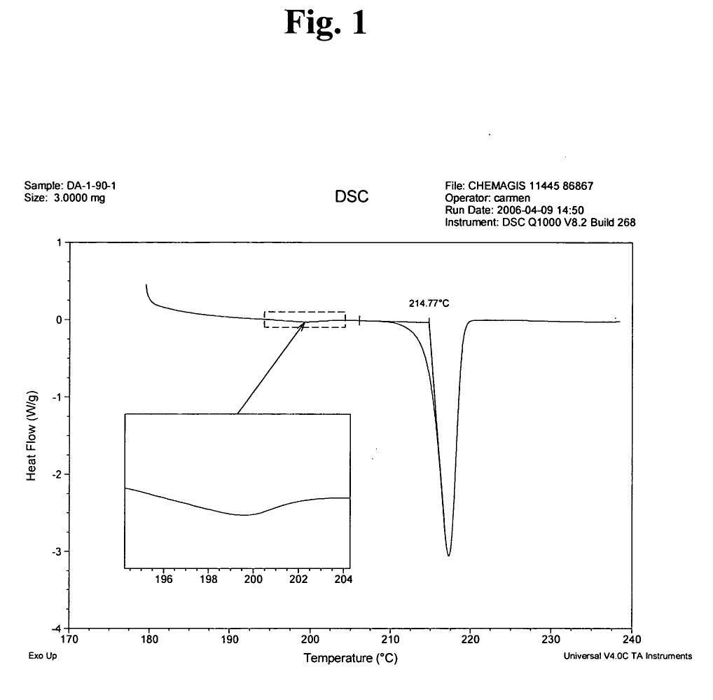

A three-necked 100 ml round bottom flask equipped with a thermometer, a reflux condenser and a magnetic stirrer was charged with 2 g of imatinib base, and mixed with 25 ml of ethanol. 276 μL Methanesulfonic acid was mixed with 5 ml ethanol. The methanesulfonic acid mixture was slowly added to imatinib base mixture. After all the methanesulfonic acid was added, the mixture was heated to reflux and maintained at that temperature for 20 minutes to produce a thick dispersion. The mixture was cooled to 65° C. and hot filtered wherein most of the material was left on the filter. The filtrate was evaporated to 50% of the volume, and the resulting solution was filtered. The second filtrate was evaporated to dryness and mixed with the first filtrate. Only minor amounts of material were obtained. The resulting material was mixed with 44 ml of ethanol, heated to reflux, and then 600 μL of water were added. The mixture was slowly cooled, but no crystallization was observed. Material from the initial step was found to contain mainly imatinib mesylate β-form with minor amount of imatinib dimesylate. FIG. 1 depicts the DSC curve of the product that was obtained as per this example, wherein an additional peak at around 200° C. is emphasized, which may be probably attributed to imatinib dimesylate form II.

### Comparative Example 2

This example illustrates an attempt to repeat example 1 of the '863 application.

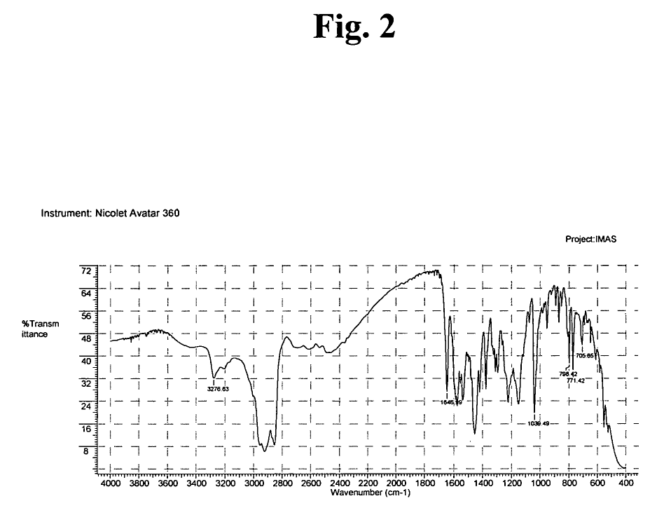

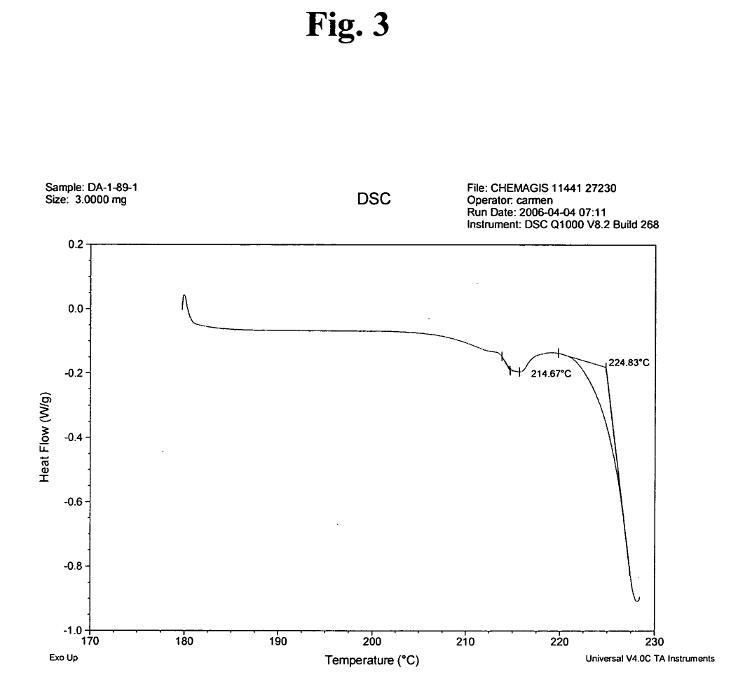

A three-necked 100 ml round bottom flask equipped with a thermometer, a reflux condenser and a magnetic stirrer was charged with 2 g of imatinib base and mixed with 25 ml of isopropyl alcohol, and the mixture was stirred for 15 minutes. 276 μL of methanesulfonic acid were mixed with 5 ml of isopropyl alcohol. The diluted methanesulfonic acid was very slowly added to the imatinib base. After all the methanesulfonic acid was added, the mixture was heated to reflux and left at that temperature during 2 hours. After 2 hours the mixture was cooled to 30° C., filtered and dried under reduced pressure. Samples were taken and analyzed by DSC and IR. The main product according to the DSC curve was imatinib dimesylate with minor amount of imatinib β-form. The infra-red spectrum of the material obtained as per this example is depicted in FIG. 2, which shows that the main product is imatinib dimesylate form I, wherein the bands at 772, 798, 1647 and 3276 cm⁻¹ are most characteristic of this form. The infra-red spectrum depicted in FIG. 2 may be compared with FIG. 11 of application '379 (of imatinib dimesylate form I), which are similar. FIG. 3 depicts the DSC curve that was carried out to the material obtained as per this example, which shows an additional peak at 214.7° C.

As can be seen from the foregoing examples, when the inventors of the present invention have tried repeating the procedure described in example 1 of the '051 patent, the main product was the β-crystalline form with small amount of imatinib dimesylate, as determined by using the infra-red and/or the DSC techniques. The inventors of the present invention have also repeated the method of example 1 of the '863 application and, based on infra-red and/or DSC techniques, obtained mainly imatinib dimesylate. Hence, it may be concluded that the processes described in example 1 of the '051 patent and example 1 of the '863 application are not reproducible.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A process for preparing crystalline imatinib mesylate in substantially pure α-form, the process comprising crystallizing imatinib mesylate from a solution comprising an organic solvent, imatinib and methanesulfonic acid dissolved therein, and seed crystals of substantially pure imatinib mesylate α-form, wherein the seed crystals are added before imatinib mesylate begins to precipitate from the solution.

2. The process of claim 1, comprising:
heating a mixture of imatinib base and an organic solvent to dissolve at least a portion of the imatinib base in the organic solvent;
seeding with crystals of imatinib mesylate α-form;
separately preparing a solution of methanesulfonic acid in the organic solvent;
gradually adding to the imatinib base solution a solution of methanesulfonic acid in the organic solvent;
allowing the mixture to cool, to precipitate crystals of imatinib mesylate in substantially pure α-form; and
isolating the precipitated crystals.

3. The process of claim 2, comprising:
heating a mixture of imatinib base and an organic solvent;
separately preparing a solution of methanesulfonic acid in the organic solvent;
gradually adding about one third of the volume of the methanesulfonic acid solution to the mixture of imatinib base and the organic solvent;
seeding the resulting mixture with seed crystals of imatinib mesylate α-form to produce a seeded mixture;
gradually adding the remaining volume of the methanesulfonic acid solution to the seeded mixture;
allowing the mixture to cool, to precipitate crystals of imatinib mesylate in substantially pure α-form; and
isolating the precipitated crystals.

4. The process of claim 2, wherein the organic solvent is methyl ethyl ketone (MEK), methyl isobutyl ketone (MIBK), cyclohexanone, 4-methylcyclohexanone, acetonitrile, or a mixture thereof.

5. The process of claim 3, wherein the molar ratio of imatinib base:methanesulfonic acid is about 1:1.

6. The process of claim 3, wherein the organic solvent is methyl ethyl ketone (MEK), methyl isobutyl ketone (MIBK), cyclohexanone, 4-methylcyclohexanone, or a mixture thereof, and the process is performed at a temperature below about 80° C.

7. The process of claim 6, wherein the process is performed at a temperature below about 70° C.

8. The process of claim 3, wherein the organic solvent is acetonitrile and the process is performed at a temperature of about 40° C. or lower.

9. The process of claim 8, wherein the process is performed at a temperature of about 15°0 C. or lower.

10. The process of claim 3, wherein the precipitated crystals are substantially free of β-form crystals based on DSC.

11. The process of claim 3, wherein at least a portion of the imatinib base is suspended in the organic solvent.

12. The process of claim 3, wherein the seed crystals are added in an amount of about 5 wt % relative to the imatinib base.

13. The process of claim 3, wherein precipitated imatinib mesylate α-form crystals have a purity equal to or greater than about 98.8%.

14. The process of claim 3, wherein precipitated imatinib mesylate α-form crystals have a purity equal to or greater than about 99.5%.

15. The process of claim 3, wherein precipitated imatinib mesylate α-form crystals are obtained in a yield greater than about 86.5%.

16. The process of claim 3, wherein precipitated imatinib mesylate α-form crystals are obtained in a yield greater than about 92%.

17. Stable, free-flowing imatinib mesylate crystals in substantially pure α-form.

18. The crystals of claim 17, substantially free of imatinib mesylate β-form crystals.

19. A pharmaceutical composition comprising a pharmaceutically acceptable carrier and a therapeutically effective amount of the crystals of claim 17.
